(19) United States
(12) Patent Application Publication
(10) Pub. No.: US 2014/0248297 A1
(43) Pub. Date: Sep. 4, 2014

(54) PREDICTORS OF RESPONSE TO IMMUNOTHERAPY

(71) Applicant: NovaRx Corporation, San Diego, CA (US)

(72) Inventors: Habib Fakhrai, La Jolla, CA (US); Daniel L. Shawler, San Diego, CA (US)

(73) Assignee: NOVARX CORPORATION, San Diego, CA (US)

(21) Appl. No.: 14/117,322

(22) PCT Filed: May 11, 2012
(86) PCT No.: PCT/US12/37629

§ 371 Date: May 20, 2014

Related U.S. Application Data
(60) Provisional application No. 61/485,523, filed on May 12, 2011.
(60) Provisional application No. 61/515,285, filed on Aug. 4, 2011.

(51) Int. Cl. G01N 33/574

(52) U.S. Cl. CPC G01N 33/57488
USPC 424/184.1; 506/9; 506/18; 530/388.2

(57) ABSTRACT

The invention is directed to a method to predict individuals who are likely to respond to immunotherapy by detecting enhanced levels of antibodies in the plasma or serum wherein the antibodies are characteristic of the response or non-response of pretested populations of subjects.

### RELATED APPLICATION

This application claims benefit of U.S. provisional applications Ser. Nos. 61/515,285 filed 4 Aug. 2011 and 61/485,523 filed 12 May 2011 which are incorporated herein by reference in their entirety.

### TECHNICAL FIELD

The invention is related to immunotherapy for the treatment of cancer. In particular, it relates to markers that permit prediction of response to immunotherapeutic treatment by assessment of blood or a fraction thereof taken from subjects early in an immunotherapeutic protocol.

### BACKGROUND ART

Immunotherapeutic treatment of cancer, whereby an attempt is made to elicit an immune response to the cancer itself has been practiced for over two decades. Such treatments typically involve administering autologous or allogeneic tumor cells bearing antigens that are expected to elicit a successful immune response to the tumor itself. Such treatments are invasive and require weeks or months of at least intermittent hospitalizations. Therefore, it would be useful to identify, early in an immunotherapeutic protocol, which subjects will successfully respond to continued treatment and for which subjects further treatment is no longer indicated using the same protocol.

One particular immunotherapeutic treatment that has been successful in a Phase II clinical trial is treatment with belagenpumatucel-L, marketed as Lucanix™. This comprises four allogeneic tumor cell lines bearing antigens common to non-small-cell lung cancer (NSCLC). These cells have been modified with an antisense vector to suppress the production of the immunosuppressant transforming growth factor-β2 (TGF-2). Only a percentage of the subjects treated, however, successfully respond. The invention provides assays which permit prediction of success or failure early in the course of treatment with this composition.

### DISCLOSURE OF THE INVENTION

It has been found that after only a few treatments with the illustrative immunotherapeutic protocol employing Lucanix®, a suite of antibodies is generated in the blood of responders that differs from the suite of antibodies raised in the blood of nonresponders. Assessing the concentrations of one or more of such antibodies found in either group allows identification of responders and nonresponders sufficiently early to be useful in making medical decisions regarding treatment.

Thus, in one aspect, the invention is directed to a method to determine the probability that a patient will respond to immunotherapy, which method comprises detecting, in the plasma or serum of said patient, elevated levels of one or more antibodies that bind to a protein selected from the proteins in Group A or a protein selected from the proteins in Group B. A patient whose plasma or serum exhibits elevated levels of one or more antibodies to a protein of Group A is identified as likely to respond to immunotherapy and a patient whose blood or serum exhibits elevated levels of an antibody that binds to a protein of Group B is identified as unlikely to respond to immunotherapy.

It has been determined that there are 22 proteins in Group A which are: EGF-like repeats and discoidin I-like domains 3 (EDIL3); cDNA clone MGC:22645 IMAGE:4700961, complete cds; DIRAS family, GTP-binding RAS-like 1 (DIRAS1); thyroglobulin (TG); solute carrier family 23 (nucleobase transporters), member 2 (SLC23A2); chromosome 10 open reading frame 81 (C10orf81); angiomotin (AMOT); Wilms tumor 1 associated protein (WTAP), transcript variant 1; serologically defined colon cancer antigen 3 (SDCCAG3), transcript variant 2; leucine rich repeat containing 8 family, member D (LRRC8D); chromosome 9 open reading frame 78 (C9orf78); solute carrier family 4 (anion exchanger), member 1, adaptor protein (SLC4A1AP); myocyte enhancer factor 2D (MEF2D); SIVA1, apoptosis-inducing factor (SIVA1), transcript variant 2; tropomodulin 2 (neuronal) (TMOD2); proline-rich coiled-coil 1 (PRRC1); microtubule-associated protein, RP/EB family, member 1 (MAPRE1); regulator of G-protein signaling 10 (RGS10), transcript variant 1; chromosome 17 open reading frame 47 (C17orf47); Janus kinase 3 (JAK3); ORM1-like 1 (S. cerevisiae) (ORMDL1); and DEP domain containing 6 (DEPDC6).

There are 30 proteins in Group B which are: upstream transcription factor 1 (USF1), transcript variant 2; polymerase (DNA-directed), delta 3, accessory subunit (POLD3); myeloid/lymphoid or mixed-lineage leukemia (trithorax homolog, Drosophila), translocated to 6 (MLLT6); SFRS protein kinase 1 (SRPK1); phosphatidylinositol binding clathrin assembly protein (PICALM), transcript variant 2; chromosome 19 open reading frame 57 (C19orf57); La ribonucleoprotein domain family, member 4 (LARP4), transcript variant 3; DnaJ (Hsp40) homolog, subfamily C, member 12 (DNAJC12), transcript variant 2; centaurin, delta 2 (CENTD2), transcript variant 1; chromosome 17 open reading frame 56 (C17orf56); MAX-like protein X (MLX), transcript variant 2; ASAP (FLJ21159); Rho GTPase activating protein 17 (ARHGAP17), transcript variant 1; mitogen-activated protein kinase 13 (MAPK13); MAP/microtubule affinity-regulating kinase 2 (MARK2), transcript variant 3; cortactin (CTTN), transcript variant 2; mitogen-activated protein kinase kinase 3 (MAP2K3), transcript variant A; protein kinase, cGMP-dependent, type II (PRKG2); Activin A receptor, type I (ACVR1); t-complex 10 (mouse) (TCP10); immunoglobulin (CD79A) binding protein 1 (IGBP1); adducin 2 (beta) (ADD2); peptidylprolyl isomerase D (cyclophilin D) (PPID); coiled-coil domain containing 72 (CCDC72); STE20-like kinase (yeast) (SLK); chromosome 1 open reading frame 62 (C1orf62); muscle, skeletal, receptor tyrosine kinase (MUSK); SH3-domain GRB2-like 2 (SH3GL2); CDC-like kinase 4 (CLK4); and tissue specific transplantation antigen P35B (TSTA3).

There are many algorithms and methods for determining whether a biomarker, such as those listed above, is elevated in a subject. Various criteria can be used depending on factors such as the number of subjects, the level of variability, and the like. One method is that illustrated herein, wherein the antibodies of Group A are determined to be at elevated levels when their concentrations are equal to or greater than the concentrations obtained by averaging a pool of such concentrations in patients that have demonstrated response to the immunotherapy as compared to averaged concentrations in a pool of subjects that are nonresponsive to the immunotherapy. Conversely, the antibodies that are members of Group B have elevated levels when their concentrations are equal to or greater than the concentrations obtained by averaging levels of the pool of patients that are nonresponsive to immunotherapy as compared to averaged concentrations in a pool of patients that have shown responsiveness.

The discovery of these antibody responses leads to several applications as the elevation of antibody levels in responders may indicate that such antibodies are effectors of successful immunotherapeutic treatment. Therefore, monoclonals that interact with the protein antigens of Group A will be useful themselves for therapeutic purposes. Further, the antigenic regions of the protein markers of Group A against which antibodies are elevated in responders will be useful in vaccines.

### BRIEF DESCRIPTION OF THE DRAWINGS

FIG. 1 is a diagram of the steps employed to determine the proteins of Groups A and B.

FIGS. 2A-2C show distributions of individual proteins in Group A among individual responders and nonresponders.

FIGS. 3A-3C show the distribution of levels of antibodies against illustrative proteins of Group B among individuals of responders and nonresponders tested.

### MODES OF CARRYING OUT THE INVENTION

The invention provides a method to assess the probability that subjects of immunotherapeutic protocols for cancer treatment will ultimately positively respond to the protocol and to determine which subjects will not respond and thus for whom no further treatment is indicated. The method is based on markers that were identified in view of experimentally determined levels of antibodies in the plasma or serum of subjects who have been subjected to immunotherapy and shown to respond or not to respond.

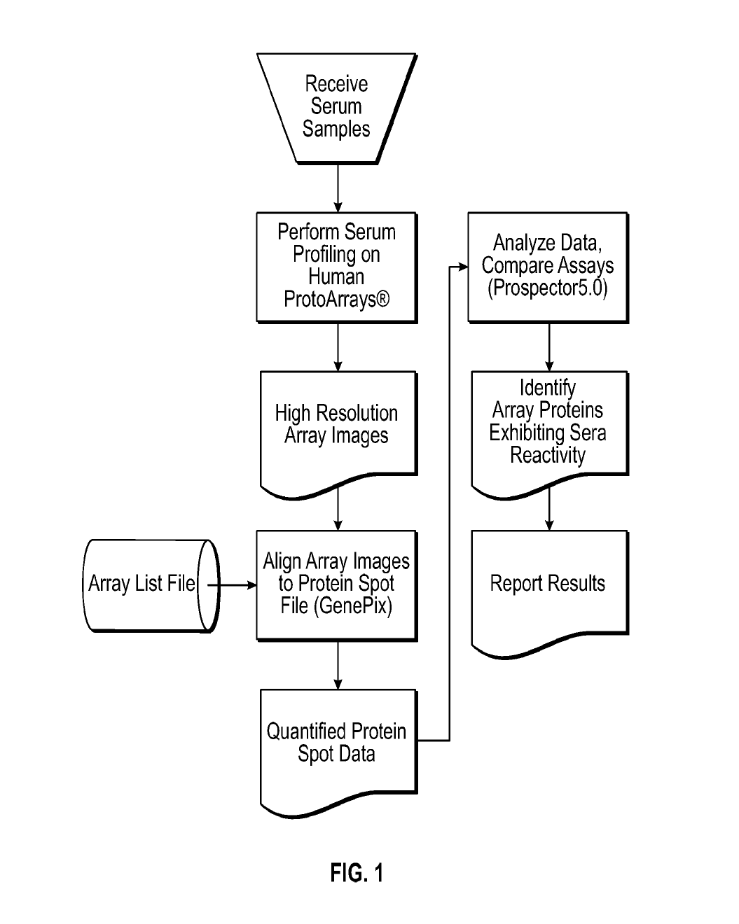

Sera were tested toward the beginning of treatment after only 10% of the treatment was complete. The test results were correlated with the ultimate responses after the course of treatment was completed and the subjects were classified as those who responded and those who did not. An outline of the process to determine the markers is shown in FIG. 1 specifically using human Prot° Arrays®, although alternative methods for assessment could have been used. The array images were quantified and analyzed to correlate which proteins in the ProtoArray® showed high or low results when tested against sera from responders or nonresponders. Using this approach, the proteins that reacted with antibodies from responders—(Group A proteins, Group A antibodies) and those that reacted with antibodies in the serum of nonresponders (i.e., antibodies and proteins of Group B) were identified. The description of members of Groups A and B is set forth above. For clarity, however, the following list uses only the abbreviations for the members of each group.

Group A:

| | | |
|---|---|---|
| EDIL3 | SDCCAG3 | MAPRE1 |
| IMAGE: 4700961 | LRRC8D | RGS10 |
| DIRAS1 | C9orf78 | cC17orf47 |
| TG | SLC4A1AP | JAK3 |
| SLC23A2 | MEF2D | ORMDL1 |
| C10orf81 | SIVA1 | DEPDC6 |
| AMOT | TMOD2 | |
| WTAP | PRRC1 | |

Group B:

| | | |
|---|---|---|
| USF1 | MLX | IGBP1 |
| POLD3 | FLJ21159 | ADD2 |
| MLLT6 | ARHGAP17 | PPID |
| SRPK1 | MAPK13 | CCDC72 |
| PICALM | MARK2 | SLK |
| C19orf57 | CTTN | C1orf62 |
| LARP4 | MAP2K3 | MUSK |
| DNAJC12 | PRKG2 | SH3GL2 |
| CENTD2 | ACVR1 | CLK4 |
| C17orf56) | TCP10 | TSTA3 |

The prognostic method of the invention requires the determination of levels of specific antibodies in the blood of the subjects wherein one or more antibodies may be determined and judgment based on the levels obtained. While the invention requires determination of one or more of such antibodies, the more antibodies that are assessed, the higher the probability of a correct conclusion. Thus, it will be advantageous, for example, to test for elevated levels of one antibody from Group A and one antibody from Group B or two antibodies from Group A or two antibodies from Group B. Groups of 3, 4, 5, 6 or 10 or 15 or more antibodies from either or both groups may also be used including testing for antibodies against all 52 proteins as well as intermediate numbers. These can be assessed using protein panels with the appropriate binding partner for the antibody to be determined.

As the method includes measurement of the concentration or level of the antibodies, the assessment can be improved by correlating the magnitude of the enhanced level of antibodies with the probability of response or nonresponse. Thus, an individual who has a particularly high level of an antibody in Group A, for example, has a higher probability of being a responder than a subject who has an elevated level, but one of less magnitude. Conversely, a subject that has a particularly high level of an antibody in Group B is less likely to respond than a subject who has an elevated level of the same antibody, but to a lesser extent.

In addition, if fewer than antibodies to all of the 52 proteins in Groups A and B are assessed, one option is to select those that appear in the greatest number of responders or nonresponders as the case may be. Another option is to test for antibodies that are found in higher percentages of the relevant group as compared to lower percentages. For example, EGF-like repeats and discoidin 1-like domains 3 (EDL3) is found in more members of the responding group than is DEP domain containing 6 (DEPDC6). Given a choice as to which antibody to determine, preference would be given to antibody against EDIL3.

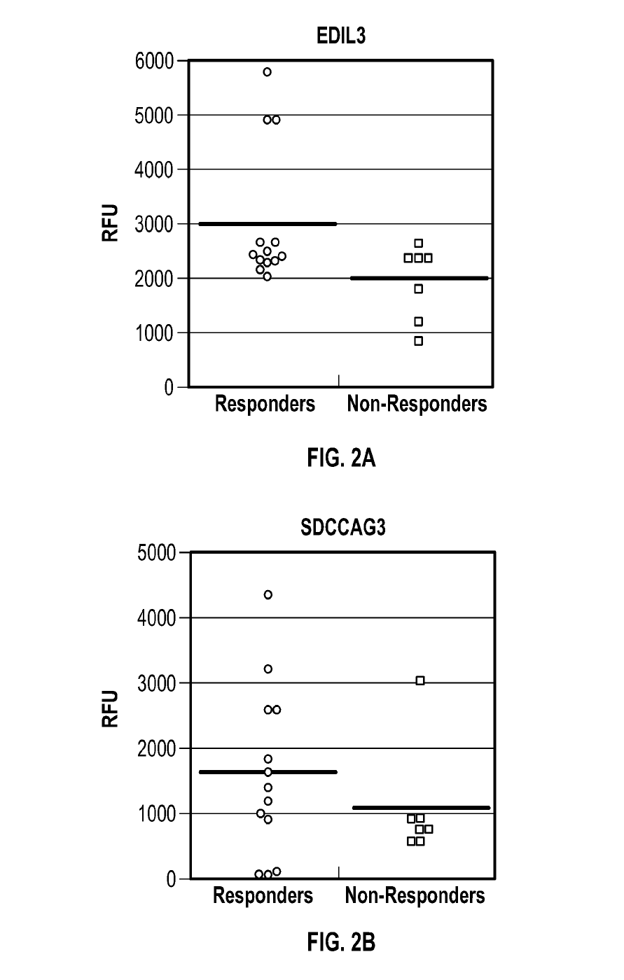

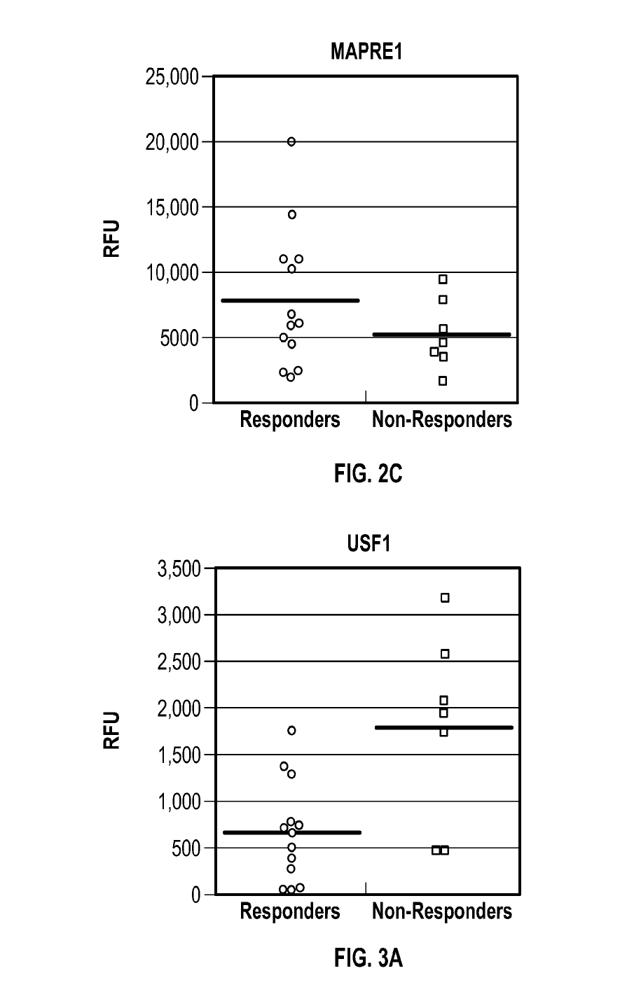

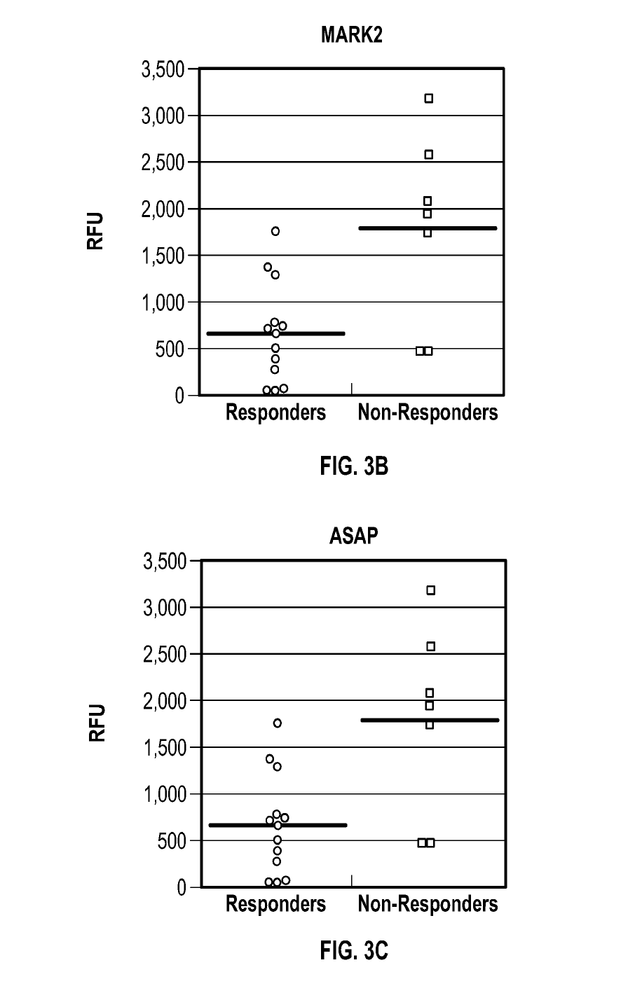

As with all correlations relating to biological data, the correlation of marker with prediction or probable outcome is not perfect. Professional judgment will be needed successfully to interpret the results. This is seen from the example below as well as the distribution plots shown in FIGS. 2 and 3. For example, FIGS. 2A-2C show the distribution of individual results for three illustrative antibodies of Group A considered to be characteristic of responders. In each case, the solid horizontal lines represent the levels averaged for responders and nonresponders respectively. Since the average of the responder group was higher than the average for nonresponders, these proteins were identified as Group A proteins—where antibody levels to these proteins could be higher in responders than in nonresponders.

In FIG. 2A, virtually all of the responders had levels of antibody that were higher than the average for the nonresponder group. All of the nonresponders had levels less than the average for the responder group. Thus, the correlation for EDIL3 is reasonably high.

However, as shown in FIG. 2B, which represents the results for SDCCAG3, several of the responders, including some who showed no evidence of antibodies against SDCCAG3, exhibited levels lower than the average for that of the nonresponders and one nonresponder had levels of antibodies significantly higher than the average for the responders. These outliers demonstrate that SDCCAG3 is somewhat less desirable as a diagnostic than EDIL3.

Finally, in FIG. 2C, showing the results for antibodies binding to MAPRE1, the distributions of the two groups appears somewhat similar, although only the responders had members with very significantly high levels of these antibodies.

Similar findings are shown in FIGS. 3A-3C for the members of Group B. As shown in FIG. 3A for antibodies against USF1, all but two of the nonresponders had antibody levels above the average for the responders and all but three higher than the nonresponder average. All of the responders had levels lower than that characteristic of the nonresponders and most even lower than that for responders. In FIG. 3B, illustrating antibodies against MARK2, two of the nonresponders had levels of antibodies against this protein lower than the average for responders and this is the case again in FIG. 3C for ASAP.

These distributions also show that there is a wide variation among subjects in each group with respect to any particular marker. Accordingly, it is desirable to use statistical methods to assess the results of an assay with regard to a particular test subject. While it is not without predictive value to measure only one antibody level and to conclude from the results that it is likely that the test subject with an elevated level of just one antibody to a protein in Group A will respond or to test just one antibody to a protein from Group B and to conclude that an individual that does not have a high level of that antibody will respond, clearly the results are greatly improved by measuring, in a test subject, more than one antibody from each panel. Microarrays are available that would make it simple to test any number of antibodies from Group A up to the total of 22 and/or any number of the antibodies in Group B up to the total of 30. Smaller numbers of antibodies from each of these groups could also readily be monitored and the results are especially favorable with regard to the response of a test subject if elevated levels of antibodies to a multiplicity of proteins in Group A is observed along with the absence of a multiplicity or low level of a multiplicity of antibodies from Group B. Clearly there are many permutations available and methods for managing the data are available in the art.

Statistical methods to manage data of this type are known, and practiced routinely. It is clear as well that some markers individually are more reliable than others and that multiplicity of markers is preferable to a single or only a few.

Based on the results with respect to the 22 markers for responders and 30 markers for nonresponders, panels of antigens are designed for convenience and maximal information in detection. Thus, antigens representing an appropriate subset of the markers of each group are designed as kits for testing subjects for prognosis. The antigens selected will be those that give the most reliable results with the least variation among responders and nonresponders and are of an appropriate number to give a statistically accurate prediction. The kits may be designed to contain, for example, individual antigens in wells of microwell plates or antigens arranged on microarrays. Because of the capacity of microarray technology, kits are designed easily to contain all 22 antigens immunoreactive with antibodies of the responders and all 30 antigens immunoreactive with antibodies of nonresponders. However, fewer than these totals may be used if desired. In addition, of course, the relevant epitopes of some or all of the antigens may be used in the panels.

Methods for assessing the levels of antibodies are well known. Typically, plasma or serum or antibody-enriched fractions thereof may be used as the samples in the assay. The assessment is typically done after only 10% of the therapeutic protocol has been conducted, or after 20% of such protocol or after 30% of such protocol has been performed. For example, if the treatment employs 20 separate sessions of administration of the autologous or allogeneic cells, the assessment may be done after two treatments, four treatments or six treatments or intermediate points. Assessment may also be done before treatment starts. Determination of the levels of antibodies may be done individually using well known immunoassays such as ELISA assays, lateral-flow assays, and the like. Alternatively, multiple groups of antibodies may be assessed by using protein arrays containing the desired number of proteins to which the presence of antibodies are to be assessed. Such arrays are commercially available or can be tailored to focus on the antibodies of interest.

The method of the invention is applicable to a variety of immunotherapeutic methods. While the markers set forth herein were determined specifically with respect to an immunotherapeutic treatment for non-small cell lung cancer, they may be used also to predict the success of alternative immunotherapies against a variety of cancers including leukemias and myelomas. Generally, the markers indicate the responsive status of the subject's immune system, and thus are applicable to immunotherapy of cancers of the breast, kidney, colon, thyroid, cervix, ovary, testes, prostate, pancreas and other organs as well as melanoma and blood-based tumors.

In addition to the design of prognostic kits, other applications of the identification of the 22 markers for responders are available.

First, while it cannot be stated with certainty, it is likely that at least some of the 22 antibodies elevated in responders are themselves responsible for the positive outcomes exhibited by these subjects. Accordingly, the antibodies themselves will be useful in treatment. The availability of the antigens immunoreactive with these antibodies permits the production of monoclonal antibodies useful in this treatment. As is understood, the monoclonal antibodies themselves may be used or immunoreactive fragments thereof, or recombinantly produced single chain versions. “Monoclonal antibodies” includes all of these. The monoclonals may be humanized or made fully human by virtue of production in, for example, XenoMouse®.

Methods to prepare monoclonal antibodies are well known in the art, as are methods for recombinant production, humanization, and the like. Methods also exist for identifying cells in the blood of the successful subjects that produce the desired antibodies with the required specificity, as set forth in PCT publication WO2005/045396. In this method, a high-throughput assay to identify individual cells that produce the desired antibody is employed and the relevant cells thus identified used as a source of nucleotide sequences encoding the desired antibody, which then may be manipulated for production of the desired monoclonals.

Thus, in one scenario, the peripheral blood cells from responders may be subjected to the screening methods set forth in the above-referenced PCT publication and screened for production of antibodies that immunoreact with the antigen sub-Group A. The encoding nucleic acids can then be isolated from these cells with or without immortalization, and used for recombinant production. Once recombinant production is made possible, various forms of the antibodies may be obtained, such as single chain Fv antibodies or even bispecific antibodies that immunoreact for example with two of the Group A markers. Thus, the monoclonal antibodies useful as passive immunotherapeutic compositions include not only complete antibodies, but may comprise only immunoreactive fragments thereof, such as Fab fragments, Fa(ab)₂ fragments, Fv antibodies and the like.

Methods are also known to identify epitopes within the antigens that immunoreact with the 22 types of antibodies elevated in the responders. Monoclonal antibodies prepared as set forth above are particularly useful in this process. However, monoclonals directly prepared from immunization with the total antigens would not be appropriate since it would be unclear whether the epitope to which these bind is, or is not, the same as that by the antibodies produced by responders. Monoclonal antibodies for epitope mapping would suitably be those prepared as described above resulting from screening the blood cells of responders and recovering the appropriate encoding nucleic acids. Epitope mapping by synthesis of overlapping peptide regions of such proteins permits identification of peptide portions of the antigens that are effective in raising the appropriate antibodies whose presence is associated with tumor regression or successful treatment. These peptides are useful as vaccines to effect immunotherapy against the tumors as well.

The epitopes thus identified can be used in the panel for detection of the appropriate antibodies and may also be used to generate additional monoclonal antibodies more precisely aimed at the desired target.

Of course, combinations of two or more such monoclonal antibodies as described above or two or more of said peptides or combinations of the monoclonal antibodies and peptides may be used as vaccines for treatment of malignancy.

The following examples are offered to illustrate but not to limit the invention.

### EXAMPLE 1

### Identification of Antibodies Characteristic of Groups A and B

Serum samples from twenty (20) non-small cell lung cancer patients treated with Lucanix® were profiled on ProtoArray® Human Protein Microarrays v4.1 containing more than 8,000 human proteins. Sera were profiled at a 1:500 dilution, utilizing one ProtoArray® Human Protein Microarray per sample. A negative control assay was run in parallel with the samples as described below. FIG. 1 shows an overview.

Assay Controls

In the negative control assay, a ProtoArray® Human Protein Microarray was treated in an identical manner to the experimental assays, except that it was incubated with buffer containing no serum prior to incubation with Alexa Fluor®647-anti-human IgG detection reagent. A small percentage (less than 0.5%) of the proteins exhibited significant signals in the negative control assay due to interaction with the detection reagent and were eliminated from the analysis. These proteins provide reference points for data acquisition and analysis. The Alexa Fluor® conjugated antibody allows proper alignment of the spot-finding software for data acquisition. An anti-biotin antibody serves as a control for another ProtoArray® application, but is recognized by the anti-human IgG antibody used for detection in this assay. Each ProtoArray® subarray contains a gradient of human IgG, which serves as a control for proper performance of the detection reagent. Anti-human IgG antibody is also spotted as a gradient in every subarray. This antibody binds to IgG present in the serum sample and serves as a control for proper assay performance.

Assay Performance

Scanner settings were selected such that maximal signals on the array were sub-saturated, thus ensuring that the full dynamic range of the scanner was utilized. Maximizing the dynamic range of the scanner without increasing background to unacceptable levels is critical. The maximum average background signal observed across samples was 442 relative fluorescence units (RFU) in one serum sample, with the cumulative average background across all samples equal to 162. Maximal signals were observed at >65,000 RFU for all of the samples, indicating a dynamic range of greater than 2 logs. These data indicate that the experiments and data acquisition were performed under optimal conditions.

Data Analysis

In contrast to DNA microarrays which generate a range of values in which the signal intensity is thought to correspond directly to the number of transcripts, protein microarrays generate data that are evaluated for the presence or absence of a significant signal. These two data types, necessitate fundamentally different statistical approaches.

Suitable biomarkers are identified in a 3-step process briefly described below.
1. Single array analysis: For each protein on each array, a series of values are calculated including background subtracted signals, Z-Score, Z-Factor, CI-P value, and replicate spot coefficient of variation
2. Group characterization: Signals for each individual protein across all samples from a given population are aligned for downstream analysis
3. Identify differences between two sample populations: Utilizing M-statistics, proteins are identified for which the differential signals between two populations result in a significant p-value

Quantitated spot files are processed using proprietary ProtoArray® Prospector software to determine which proteins interact with the samples. The software performs all aspects of the analysis, including background correction, normalization and M-statistic calculations of significance.

The output from the comparative analysis performed by ProtoArray® Prospector includes information on the number of patients in each population that exhibited an immune response against each ProtoArray® protein that was above the M-statistic threshold established for that protein. These numbers correspond to the “Count” shown in Tables 1 and 2 below. Additional thresholds were imposed requiring that signals be at least 500 relative fluorescence units (RFU), and a minimum signal difference of 200 RFU must be observed between samples from two populations in order for a particular sample to be included in the M-statistic Count. The “Cutoff” value reported in Tables 1 and 2 below corresponds to the value 200 RFU above the M-statistic signal threshold established for that protein. The maximal population prevalence is reported for each protein based on the sample size and the M-statistic Count. These numbers correspond to the “Prevalence” values reported in Tables 1 and 2 below. All data were normalized using the Robust Linear Model (RLM) prior to M-statistics analysis.

Proteins were identified as candidate autoantigens if they met the following criteria:
The M-statistic signal threshold (“Cutoff”) was greater than 500 RFU.
The p-value threshold was less than 0.1.

### Identification of Candidate Biomarkers: Elevated Signals in the Sera from Patients Classified as Responders to Vaccine Treatment

Twenty-two (22) ProtoArray® proteins exhibited elevated interactions with serum autoantibodies in responder sera relative to nonresponder sera that met the threshold criteria described above. Group (Grp) 1 represents responders and Group 2 represents nonresponders. These 22 proteins, ranked by P-value, are presented in Table 1.

TABLE 1

| | | Count | | Prevalence | | | | |
|---|---|---|---|---|---|---|---|---|
| | Ultimate | Grp | Grp | Grp | Grp | | Cut- | |
| Database ID | ORF ID | 1 | 2 | 1% | 2% | P-Value | off | Protein Description |
| BC053656.1 | IOH28981 | 13 | 4 | 93 | 56 | 3.07E−02 | 1975 | EGF-like repeats and discoidin I-like |
| | | | | | | | | domains 3 (EDIL3) |
| BC030813.1 | IOH23055 | 12 | 3 | 87 | 44 | 3.07E−02 | 3784 | cDNA clone MGC: 22645 |
| | | | | | | | | IMAGE: 4700961, complete cds |
| NM_145173.1 | IOH22396 | 6 | 0 | 47 | 11 | 4.43E−02 | 1281 | DIRAS family, GTP-binding RAS- |
| | | | | | | | | like 1 (DIRAS1) |
| thyroglobulin | | 6 | 0 | 47 | 11 | 4.43E−02 | 3736 | thyroglobulin (TG) |
| BC013112.2 | IOH21474 | 6 | 0 | 47 | 11 | 4.43E−02 | 1158 | solute carrier family 23 (nucleobase |
| | | | | | | | | transporters), member 2 (SLC23A2) |
| BC036365.1 | IOH22309 | 6 | 0 | 47 | 11 | 4.43E−02 | 983 | chromosome 10 open reading |
| | | | | | | | | frame 81 (C10orf81) |
| NM_133265.2 | IOH38152 | 6 | 0 | 47 | 11 | 4.43E−02 | 6100 | angiomotin (AMOT) |
| NM_004906.3 | IOH40770 | 6 | 0 | 47 | 11 | 4.43E−02 | 1751 | Wilms tumor 1 associated protein |
| | | | | | | | | (WTAP), transcript variant 1 |
| NM_006643.2 | IOH12301 | 8 | 1 | 60 | 22 | 5.79E−02 | 1139 | serologically defined colon cancer |
| | | | | | | | | antigen 3 (SDCCAG3), transcript |
| | | | | | | | | variant 2 |
| BC009486.1 | IOH22946 | 8 | 1 | 60 | 22 | 5.79E−02 | 2176 | leucine rich repeat containing 8 |
| | | | | | | | | family, member D (LRRC8D) |
| BC017570.1 | IOH27880 | 8 | 1 | 60 | 22 | 5.79E−02 | 588 | chromosome 9 open reading frame 78 |
| | | | | | | | | (C9orf78) |
| NM_018158.1 | IOH38323 | 5 | 0 | 40 | 11 | 8.30E−02 | 723 | solute carrier family 4 (anion |
| | | | | | | | | exchanger), member 1, adaptor protein |
| | | | | | | | | (SLC4A1AP) |
| BC040949.1 | IOH26268 | 5 | 0 | 40 | 11 | 8.30E-02 | 2547 | myocyte enhancer factor 2D (MEF2D) |
| NM_021709.1 | IOH21450 | 5 | 0 | 40 | 11 | 8.30E-02 | 1143 | SIVA1, apoptosis-inducing factor |
| | | | | | | | | (SIVA1), transcript variant 2 |
| NM_014548.2 | IOH39971 | 5 | 0 | 40 | 11 | 8.30E−02 | 2200 | tropomodulin 2 (neuronal) (TMOD2) |
| BC017066.1 | IOH11186 | 5 | 0 | 40 | 11 | 8.30E−02 | 1272 | proline-rich coiled-coil 1 (PRRC1) |
| NM_012325.1 | IOH41294 | 5 | 0 | 40 | 11 | 8.30E−02 | 9776 | microtubule-associated protein, RP/EB |
| | | | | | | | | family, member 1 (MAPRE1) |
| NM_001005339.1 | IOH13018 | 5 | 0 | 40 | 11 | 8.30E−02 | 507 | regulator of G-protein signaling 10 |
| | | | | | | | | (RGS10), transcript variant 1 |
| BC022189.2 | IOH22420 | 5 | 0 | 40 | 11 | 8.30E−02 | 958 | chromosome 17 open reading frame |
| | | | | | | | | 47 (C17orf47) |
| PV3855 | | 5 | 0 | 40 | 11 | 8.30E−02 | 539 | Janus kinase 3 (JAK3) |
| NM_016467.1 | IOH7173 | 5 | 0 | 40 | 11 | 8.30E−02 | 614 | ORM1-like 1 (S. cerevisiae) |
| | | | | | | | | (ORMDL1) |
| BC012040.1 | IOH9723 | 5 | 0 | 40 | 11 | 8.30E−02 | 1080 | DEP domain containing 6 (DEPDC6) |

The candidate biomarkers span a diverse range of biochemical functions or cellular roles and belong to variety of categories. Many of these proteins, however, have links to various cancers, including EDIL3, SLC23A2, AMOT, WTAP, MEF2D, MAPRE1, RGS10 and JAK3. EDIL3 and WTAP are overexpressed in liver cancer and breast cancer, respectively, and both may play a role in tumor cell migration. MAPRE1 is overexpressed in esophageal squamous cell carcinoma and interacts with the adenomatous polyposis coli (APC) tumor suppressor protein. Mutations in APC are associated with certain intestinal tumors, and inhibitors of JAK3 are being evaluated as anti-cancer drugs that prevent development of these tumors.

### Identification of Candidate Biomarkers: Elevated Signals in the Sera from Patients Classified as Nonresponders to Vaccine Treatment

Thirty (30) ProtoArray® proteins exhibited elevated interactions with serum autoantibodies in nonresponder sera relative to responder sera that met the threshold criteria described above. Again, Group 1 represents responders and Group 2 represents nonresponders. These 30 proteins, ranked by P-value, are presented in Table 2.

TABLE 2

| | | Count | | Prevalence | | | | |
|---|---|---|---|---|---|---|---|---|
| | Ultimate | Grp | Grp | Grp | Grp | | Cut- | |
| Database ID | ORF ID | 1 | 2 | 1% | 2% | P-Value | off | Protein Description |
| NM_207005.1 | IOH45744 | 0 | 4 | 7 | 56 | 7.22E−03 | 1941 | upstream transcription factor 1 |
| | | | | | | | | (USF1), transcript variant 2 |
| NM_006591.1 | IOH41599 | 0 | 4 | 7 | 56 | 7.22E−03 | 745 | polymerase (DNA-directed), delta 3, |
| | | | | | | | | accessory subunit (POLD3) |
| BC064612.1 | IOH39905 | 3 | 6 | 27 | 78 | 1.24E−02 | 2094 | myeloid/lymphoid or mixed-lineage |
| | | | | | | | | leukemia (trithorax homolog, |
| | | | | | | | | Drosophila); translocated to 6 |
| | | | | | | | | (MLLT6) |
| PV4215 | | 0 | 3 | 7 | 44 | 3.07E−02 | 1195 | SFRS protein kinase 1 (SRPK1) |
| NM_001008660.1 | IOH39850 | 0 | 3 | 7 | 44 | 3.07E−02 | 1321 | phosphatidylinositol binding clathrin |
| | | | | | | | | assembly protein (PICALM), |
| | | | | | | | | transcript variant 2 |
| BC012945.1 | IOH25802 | 0 | 3 | 7 | 44 | 3.07E−02 | 6599 | chromosome 19 open reading |
| | | | | | | | | frame 57 (C19orf57) |
| NM_199190.1 | IOH42760 | 0 | 3 | 7 | 44 | 3.07E−02 | 2496 | La ribonucleoprotein domain family, |
| | | | | | | | | member 4 (LARP4), transcript variant 3 |
| NM_201262.1 | IOH41260 | 0 | 3 | 7 | 44 | 3.07E−02 | 15145 | DnaJ (Hsp40) homolog, subfamily C, |
| | | | | | | | | member 12 (DNAJC12), transcript |
| | | | | | | | | variant 2 |
| NM_139181.1 | IOH42674 | 0 | 3 | 7 | 44 | 3.07E−02 | 757 | centaurin, delta 2 (CENTD2), |
| | | | | | | | | transcript variant 1 |
| NM_144679.1 | IOH40679 | 0 | 3 | 7 | 44 | 3.07E−02 | 2741 | chromosome 17 open reading |
| | | | | | | | | frame 56 (C17orf56) |
| NM_198204.1 | IOH9777 | 0 | 3 | 7 | 44 | 3.07E−02 | 759 | MAX-like protein X (MLX), transcript |
| | | | | | | | | variant 2 |
| NM_024826.1 | IOH42194 | 0 | 3 | 7 | 44 | 3.07E−02 | 8487 | ASAP (FLJ21159) |
| NM_001006634.1 | IOH44197 | 1 | 4 | 13 | 56 | 3.07E−02 | 1550 | Rho GTPase activating protein 17 |
| | | | | | | | | (ARHGAP17), transcript variant 1 |
| NM_002754.3 | IOH3435 | 1 | 4 | 13 | 56 | 3.07E−02 | 880 | mitogen-activated protein kinase 13 |
| | | | | | | | | (MAPK13) |
| PV3878 | | 4 | 7 | 33 | 89 | 4.43E−02 | 633 | MAP/microtubule affinity-regulating |
| | | | | | | | | kinase 2 (MARK2), transcript |
| | | | | | | | | variant 3 |
| NM_138565.1 | IOH6227 | 7 | 7 | 53 | 89 | 4.43E−02 | 483 | cortactin (CTTN), transcript variant 2 |
| PV3662 | | 2 | 5 | 20 | 67 | 5.21E−02 | 1104 | mitogen-activated protein kinase |
| | | | | | | | | kinase 3 (MAP2K3), transcript variant |
| | | | | | | | | A |
| PV3973 | | 3 | 5 | 27 | 67 | 5.21E−02 | 2070 | protein kinase, cGMP-dependent, type |
| | | | | | | | | II (PRKG2) |
| PV4877 | | 3 | 5 | 27 | 67 | 5.21E−02 | 769 | Activin A receptor, type I (ACVR1) |
| BC063451.1 | IOH39854 | 4 | 6 | 33 | 78 | 5.79E−02 | 1776 | t-complex 10 (mouse) (TCP10) |
| NM_001551.1 | IOH3828 | 5 | 6 | 40 | 78 | 5.79E−02 | 1143 | immunoglobulin (CD79A) binding |
| | | | | | | | | protein 1 (IGBP1) |
| BC065525.1 | IOH40477 | 2 | 4 | 20 | 56 | 7.77E−02 | 2318 | adducin 2 (beta) (ADD2) |
| NM_005038.1 | IOH22406 | 6 | 7 | 47 | 89 | 8.30E−02 | 780 | peptidylprolyl isomerase D |
| | | | | | | | | (cyclophilin D) (PPID) |
| NM_015933.1 | IOH3769 | 7 | 7 | 53 | 89 | 8.30E−02 | 1057 | coiled-coil domain containing 72 |
| | | | | | | | | (CCDC72) |
| PV3830 | | 7 | 7 | 53 | 89 | 8.30E−02 | 733 | STE20-like kinase (yeast) (SLK) |
| NM_152763.2 | IOH22315 | 7 | 7 | 53 | 89 | 8.30E−02 | 857 | chromosome 1 open reading frame 62 |
| | | | | | | | | (C1orf62) |
| PV3834 | | 8 | 7 | 60 | 89 | 8.30E−02 | 780 | muscle, skeletal, receptor tyrosine |
| | | | | | | | | kinase (MUSK) |
| BC032825.2 | IOH26816 | 8 | 7 | 60 | 89 | 8.30E−02 | 845 | SH3-domain GRB2-like 2 (SH3GL2) |
| PV3839 | | 8 | 7 | 60 | 89 | 8.30E−02 | 916 | CDC-like kinase 4 (CLK4) |
| NM_003313.2 | IOH4998 | 8 | 7 | 60 | 89 | 8.30E−02 | 521 | tissue specific transplantation antigen |
| | | | | | | | | P35B (TSTA3) |

Many of the candidate biomarkers that were identified in nonresponder sera can be grouped into several categories that span diverse biological processes:
Protein kinases, including, SRPK1, MAPK13, MAP2K3, MARK2, PRKG2, SLK, MUSK, and CLK4. MAP2K3 is reported to be upregulated and aberrantly methylated in one lung adenocarcinoma cell line. SRPK1 is overexpressed in breast, colon, and pancreatic cancers, while MARK2 expression is increased in melanoma.
Proteins involved in protein folding, including DNAJC12 and PPID.
Transcription factors, including USF1, MLLT6, and MLX. Levels of USF1 expression have been shown to be reduced in non-small cell lung carcinomas relative to non-tumorous tissue.
Proteins with roles related to cell cycle regulation, including ASAP. ASAP is required for proper mitotic progression and is a substrate of the centrosomal kinase Aurora A. Aurora A is overexpressed in numerous cancers, suggesting that ASAP may represent an additional target for anti-cancer drugs.
Apoptosis associated proteins, including CENTD2, PPID, and CCDC72. CENTD2 may play a role in inducing apoptosis of cancer cells, while overexpression of PPID may suppress apoptosis of cancer cells.

1. A method to determine the probability that a patient will respond to immunotherapy, which method comprises:
detecting in the plasma or serum of said patient elevated levels of one or more antibodies that bind to a protein selected from the proteins in Group A or a protein selected from the proteins in Group B
wherein a patient whose plasma or serum exhibits elevated levels of one or more antibodies to the protein of Group A is identified as likely to respond to immunotherapy and a patient whose blood or serum exhibits elevated levels of an antibody that binds to a protein of Group B is identified as unlikely to respond to immunotherapy,
wherein the proteins in Group A are:
EGF-like repeats and discoidin I-like domains 3 (EDIL3); cDNA clone MGC:22645 IMAGE:4700961, complete cds; DIRAS family, GTP-binding RAS-like 1 (DIRAS1); thyroglobulin (TG); solute carrier family 23 (nucleobase transporters), member 2 (SLC23A2); chromosome 10 open reading frame 81 (C10orf81); angiomotin (AMOT); Wilms tumor 1 associated protein (WTAP), transcript variant 1; serologically defined colon cancer antigen 3 (SDCCAG3), transcript variant 2; leucine rich repeat containing 8 family, member D (LRRC8D); chromosome 9 open reading frame 78 (C9orf78); solute carrier family 4 (anion exchanger), member 1, adaptor protein (SLC4A1AP); myocyte enhancer factor 2D (MEF2D); SIVA1, apoptosis-inducing factor (SIVA1), transcript variant 2; tropomodulin 2 (neuronal) (TMOD2); proline-rich coiled-coil 1 (PRRC1); microtubule-associated protein, RP/EB family, member 1 (MAPRE1); regulator of G-protein signaling 10 (RGS10), transcript variant 1; chromosome 17 open reading frame 47 (C17orf47); Janus kinase 3 (JAK3); ORM1-like 1 (S. cerevisiae) (ORMDL1); and DEP domain containing 6 (DEPDC6),
and the proteins in Group B are:
upstream transcription factor 1 (USF1), transcript variant 2; polymerase (DNA-directed), delta 3, accessory subunit (POLD3); myeloid/lymphoid or mixed-lineage leukemia (trithorax homolog, Drosophila), translocated to 6 (MLLT6); SFRS protein kinase 1 (SRPK1); phosphatidylinositol binding clathrin assembly protein (PICALM), transcript variant 2; chromosome 19 open reading frame 57 (C19orf57); La ribonucleoprotein domain family, member 4 (LARP4), transcript variant 3; DnaJ (Hsp40) homolog, subfamily C, member 12 (DNAJC12), transcript variant 2; centaurin, delta 2 (CENTD2), transcript variant 1; chromosome 17 open reading frame 56 (C17orf56); MAX-like protein X (MLX), transcript variant 2; ASAP (FLJ21159); Rho GTPase activating protein 17 (ARHGAP17), transcript variant 1; mitogen-activated protein kinase 13 (MAPK13); MAP/microtubule affinity-regulating kinase 2 (MARK2), transcript variant 3; cortactin (CTTN), transcript variant 2; mitogen-activated protein kinase kinase 3 (MAP2K3), transcript variant A; protein kinase, cGMP-dependent, type II (PRKG2); Activin A receptor, type I (ACVR1); t-complex 10 (mouse) (TCP10); immunoglobulin (CD79A) binding protein 1 (IGBP1); adducin 2 (beta) (ADD2); peptidylprolyl isomerase D (cyclophilin D) (PPID); coiled-coil domain containing 72 (CCDC72); STE20-like kinase (yeast) (SLK); chromosome 1 open reading frame 62 (C1orf62); muscle, skeletal, receptor tyrosine kinase (MUSK); SH3-domain GRB2-like 2 (SH3GL2); CDC-like kinase 4 (CLK4); and tissue specific transplantation antigen P35B (TSTA3).

2. The method of claim 1 wherein
(a) with respect to Group A, an elevated level is defined as a concentration equal to or greater than the concentration obtained by averaging concentrations in a pool of patients that have demonstrated response to immunotherapy as compared to concentrations in a pool that is nonresponsive to immunotherapy and
(b) with respect to Group B, an elevated level is defined as a concentration equal to or greater than the concentration obtained by the averaging concentrations a pool of patients that is nonresponsive to immunotherapy as compared to concentrations in a pool of patients that have shown responsiveness to immunotherapy.

3. The method of claim 1 wherein said detecting is of at least two antibodies that bind a protein of Group A and at least two antibodies that bind a protein of Group B.

4. The method of claim 3 wherein said detecting is of at least five antibodies that bind a protein of Group A and at least five antibodies that bind a protein of Group B.

5. The method of claim 4 wherein a protein array is employed to detect said antibodies.

6. A kit for performing the method of claim 1 which kit comprises at least three antigens of Group A and three antigens of Group B or fragments thereof immunoreactive with said antibodies.

7. The kit of claim 6 wherein said proteins or fragments are deposited in an array.

8. A method to prepare a medicament for treatment of malignancy in a subject which method comprises preparing monoclonal antibodies immunoreactive with a protein of Group A.

9. A medicament prepared by the method of claim 8.

10. A method to prepare a vaccine for amelioration of malignancy in a subject which method comprises identifying at least one region of at least one of the antigens immunoreactive with an antibody of Group A and synthesizing a peptide corresponding to said region.

11. A vaccine prepared by the method of claim 10.
